(19) United States
(12) Patent Application Publication
(10) Pub. No.: US 2026/0014367 A1
(43) Pub. Date: Jan. 15, 2026

(54) THERAPEUTIC DRESSING

(71) Applicant: BIRMINGHAM HAND AND NERVE LTD, BIRMINGHAM (GB)

(72) Inventors: TAHSEEN CHAUDHRY, BIRMINGHAM (GB); AWNI AL-JAYYOUSSI, BIRMINGHAM (GB)

(21) Appl. No.: 19/262,854

(22) Filed: Jul. 8, 2025

Related U.S. Application Data
(60) Provisional application No. 63/668,865, filed on Jul. 9, 2024.

(51) Int. Cl. A61N 1/04; A61F 13/00; A61F 13/0246; A61N 1/02; A61N 1/36

(52) U.S. Cl. CPC A61N 1/0468; A61F 13/025; A61N 1/025; A61N 1/0492; A61N 1/36031; A61N 1/36034; A61F 2013/00919

(57) ABSTRACT

Therapeutic dressings for wounds are provided, as well as methods of using the same and methods of fabricating the same. A therapeutic dressing can include a central non-adherent portion, an adherent portion surrounding the central non-adherent portion, a sensor, at least one stimulating electrode, and a circuit in operable communication with the sensor and the at least one stimulating electrode. The electrode(s) can deliver an electrical stimulus to one or more nerves through the skin of a patient/user. The sensor can be, for example, an electromyography (EMG) sensor, and the therapeutic dressing can be configured such that the sensor and the electrode(s) are in direct physical contact with the skin during use.

### CROSS-REFERENCE TO RELATED APPLICATION

This application claims the benefit of U.S. Provisional Application Ser. No. 63/668,865, filed Jul. 9, 2024, the disclosure of which is hereby incorporated by reference in its entirety, including all figures, tables, and drawings.

### BACKGROUND

Peripheral nerve injuries are common and can result in significant functional deficits. Nerve repair/reconstruction or decompression surgery is often performed to restore nerve function. However, the recovery process can be slow, and there is a need for adjunct therapies that can accelerate recovery and improve outcomes. The following documents describe nerve stimulation systems, but each has limitations-U.S. Pat. No. 11,478,631, U.S. Patent Application Publication No. 2021/0268276, U.S. Pat. Nos. 11,344,719, 11,596,792, U.S. Patent Application Publication No. 2017/0136265, U.S. Patent Application Publication No. 2019/366083, U.S. Patent Application Publication No. 2017/0080255, International Patent Application Publication No. WO02/089911, International Patent Application Publication No WO02/089911, and CN20918791.

### BRIEF SUMMARY

In order to address the limitations of the related art, embodiments of the subject invention provide novel and advantageous therapeutic dressings for wounds, as well as methods of using the same and methods of fabricating the same. A therapeutic dressing can include a central non-adherent portion and an adherent portion surrounding the central non-adherent portion. The adherent portion can comprise an adhesive on one side thereof (i.e., the side configured to be placed on the skin). The therapeutic dressing can be made of a soft material, such as a soft polymer material. The central non-adherent portion and the adherent portion can comprise the same material or can comprise different materials. The therapeutic dressing can include a sensor, at least one stimulating electrode, and a circuit in operable communication with the at least one stimulating electrode and/or the sensor, the circuit being configured to control the at least one stimulating electrode and/or the sensor. The at least one stimulating electrode can deliver an electrical stimulus (e.g., a constant electrical stimulus) to one or more nerves through the skin. The sensor can be, for example, an electromyography (EMG) sensor, and the therapeutic dressing can be configured such that the sensor and/or the electrode(s) are in direct physical contact with the skin during use. In some cases, the at least one stimulating electrode, the sensor, and the circuit can be provided on a separate layer (i.e., on an upper layer) from the non-adherent portion and the adherent portion and can be joined thereto by a peelable adhesive, such that a user can peel off the upper layer (with the at least one stimulating electrode, the sensor, and the circuit) while leaving the lower layer on the wound as a wound dressing.

In an embodiment, a therapeutic dressing can comprise: a central non-adherent portion; an adherent portion around the central non-adherent portion and comprising an adhesive; at least one stimulating electrode (e.g., a positive stimulating electrode and a negative stimulating electrode); a sensor; and an integrated circuit (IC) in operable communication with, and configured to control, the at least one stimulating electrode. The therapeutic dressing can be configured to provide electrical stimulation to a nerve of a user (e.g., a patient, such as a patient who has recently had nerve surgery) during use via the at least one stimulating electrode. The IC can be configured to such that the nerve receives the same amount of stimulating current regardless of a size of the user or a resistance of skin or tissue of the user. The IC can comprise a microcontroller, a power source, and a current-controlled voltage pulse generator configured to provide the same amount of stimulating current to the nerve regardless of the size of the user or the resistance of the skin or tissue of the user. The therapeutic dressing can be configured to provide the electrical stimulation to the nerve for a predetermined amount of time before becoming inert. The predetermined amount of time can be based on characteristics of a wound of the nerve of the user and/or characteristics of the user. The predetermined amount of time can be in a range of, for example, from 2 minutes to 180 minutes (e.g., from 30 minutes to 90 minutes, or 60 minutes or about 60 minutes). The therapeutic dressing can further comprise a first layer of a dressing material and a second layer of the dressing material. The IC can be sandwiched between the first layer of the dressing material and the second layer of the dressing material; the sensor can be exposed out of the first layer of the dressing material (e.g., the layer configured to face the skin of the user during use); the at least one stimulating electrode can be exposed out of the first layer of the dressing material; and/or the dressing material can be electrically insulating. The therapeutic dressing can be configured such that the sensor is in direct physical contact with skin of the user during use. The therapeutic dressing can be configured such that the sensor is disposed over a muscle of the user during use. The therapeutic dressing can be configured such that the at least one stimulating electrode is in direct physical contact with skin of the user during use. The sensor can be an EMG sensor. The therapeutic dressing can further comprise a perforated border between the central non-adherent portion and the adherent portion. The therapeutic dressing can further comprise an indicator configured to indicate a function of the IC and/or the at least one stimulating electrode during use. The indicator can comprise at least one light (e.g., at least one light-emitting diode (LED)). The IC, the sensor, and the at least one stimulating electrode, and/or the indicator (if present) can be disposed in the non-adherent portion. The therapeutic dressing can be a single-use, disposable dressing. The IC can comprise at least one battery and a voltage multiplier (e.g., a voltage multiplier comprising a plurality of capacitors and a plurality of diodes). The IC can be in operable communication with, and configured to control, the sensor. The central non-adherent portion can comprise a same dressing material as the adherent portion (or can alternatively comprise a different dressing material as the adherent portion). The therapeutic dressing can comprise a dressing material that is soft (i.e., not rigid). The dressing material can be, for example, a (soft) polymer dressing material. The IC can comprise a wireless communication module (e.g., a Bluetooth integrated circuit) configured to enable remote modification of one or more stimulation parameters of the electrical stimulation. The IC can comprise a digital signal processor (DSP) configured to condition one or more physiological input signals. The DSP can be configured to reduce or eliminate junction-reference artifacts.

In another embodiment, a method for providing electrical stimulation to a nerve of a user can comprise: applying, to skin of the user, a therapeutic dressing having any or all of the features discussed herein (including in the previous paragraph); and using (or operating) the therapeutic dressing to provide the electrical stimulation to the nerve for a predetermined amount of time. The predetermined amount of time can be based on characteristics of a wound of the nerve of the user and/or characteristics of the user. The predetermined amount of time can be in a range of, for example, from 2 minutes to 180 minutes (e.g., from 30 minutes to 90 minutes, or 60 minutes or about 60 minutes). The method can further comprise disposing of the therapeutic dressing after a single use of the therapeutic dressing providing the electrical stimulation to the nerve for the predetermined amount of time.

In another embodiment, a method of monitoring a post-operative treatment site can comprise: applying to the post-operative treatment site a therapeutic dressing (e.g., having any or all of the features discussed herein (including in the paragraph before the previous paragraph)) (e.g., the therapeutic dressing can comprise a circuit (e.g., an IC) and at least one sensor); receiving a physiological signal from the (at least one) sensor of the therapeutic dressing; transmitting the physiological signal wirelessly to a remote device; and adjusting (e.g., via the remote device) one or more stimulation parameters of the therapeutic dressing based on the physiological signal.

In another embodiment, a method for providing electrical stimulation to a nerve of a user can comprise: applying, to skin of the user, a therapeutic dressing (e.g., having any or all of the features discussed herein (including in the second paragraph of this section)); receiving and utilizing EMG signal feedback to confirm neural stimulation; refining and/or adjusting stimulation parameters of the therapeutic dressing, based on the EMG signal feedback, to deliver and/or maintain optimal stimulation according to a predetermined protocol.

### BRIEF DESCRIPTION OF DRAWINGS

FIG. 1 shows a therapeutic dressing, according to an embodiment of the subject invention.

FIG. 2 shows a circuit diagram of an integrated circuit that can be used with a therapeutic dressing, according to an embodiment of the subject invention.

### DETAILED DESCRIPTION

Embodiments of the subject invention provide novel and advantageous therapeutic dressings for wounds, as well as methods of using the same and methods of fabricating the same. A therapeutic dressing can include a central non-adherent portion and an adherent portion surrounding the central non-adherent portion. The adherent portion can comprise an adhesive on one side thereof (i.e., the side configured to be placed on the skin). The therapeutic dressing can be made of a soft material, such as a soft polymer material. The central non-adherent portion and the adherent portion can comprise the same material or can comprise different materials. The therapeutic dressing can include a sensor, at least one stimulating electrode, and a circuit in operable communication with the at least one stimulating electrode and/or the sensor, the circuit being configured to control the at least one stimulating electrode and/or the sensor. The at least one stimulating electrode can deliver an electrical stimulus (e.g., a constant electrical stimulus) to one or more nerves through the skin. The sensor can be, for example, an electromyography (EMG) sensor, and the therapeutic dressing can be configured such that the sensor and/or the electrode(s) are in direct physical contact with the skin during use.

In some cases, the at least one stimulating electrode, the sensor, and the circuit can be provided on a separate layer (i.e., on an upper layer) from the non-adherent portion and the adherent portion and can be joined thereto by a peelable adhesive, such that a user can peel off the upper layer (with the at least one stimulating electrode, the sensor, and the circuit) while leaving the lower layer on the wound as a wound dressing.

A therapeutic dressing can be applied onto a wound (e.g., after a surgery, such as peripheral nerve surgery), and can comprise a dressing material, a circuit, a sensor, and at least one stimulating electrode. The dressing material and/or the other components can be disposable, such that the therapeutic dressing can be disposable. The circuit can control the electrode(s) to deliver an electrical stimulus (e.g., a constant electrical stimulus) to a nerve of interest through the skin of the patient/user. The circuit can be configured to alter the applied voltage such that the nerve receives the same amount of stimulating voltage regardless of patient size and/or local skin/tissue resistance. The circuit can comprise a microcontroller, a power source, and a current-controlled voltage pulse generator that guarantees the same level of current/voltage delivered to the nerve regardless of patient size and/or local skin/tissue resistance. The therapeutic dressing can be configured to maintain the electrical stimulation for a predetermined amount of time before stopping and/or before the electrode(s) and/or circuit become inert. The duration of stimulation can be predetermined (e.g., based on the specific surgical procedure and/or patient characteristics).

In some embodiments, the therapeutic dressing can be used as an adjunct to peripheral nerve surgery following repair/reconstruction or decompression of nerves. The dressing can provide a constant electrical stimulus to the nerve through the skin and can alter the applied voltage such that the nerve receives the same amount of stimulating current/voltage regardless of patient size and/or local skin/tissue resistance. The amount of nerve stimulation can be predetermined, and the therapeutic dressing can be designed to be a single-use item that replaces a traditional post-operative dressing following nerve surgery. The therapeutic dressing can maintain stimulation for a set (i.e., predetermined) number of minutes before becoming inert. The therapeutic dressing can be disposable and can sit within and/or be a part of a regular adherent post-surgical wound dressing (referred to herein as the adherent portion).

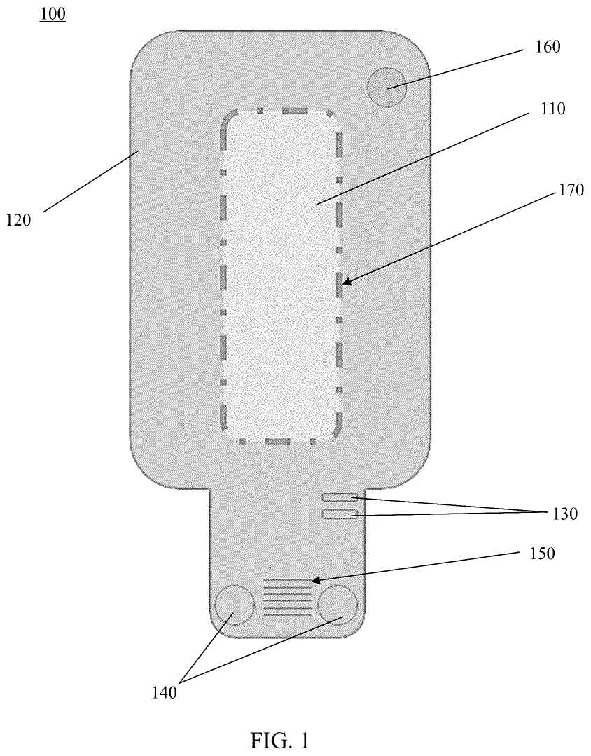

FIG. 1 shows a therapeutic dressing, according to an embodiment of the subject invention. FIG. 1 shows the dressing from the bottom (i.e., the surface that would come into contact with the skin). Referring to FIG. 1, the therapeutic dressing 100 can include a central non-adherent portion 110 and an adherent portion 120. In some embodiments, the central non-adherent portion 110 and the adherent portion 120 can be the same material, such that they form one continuous dressing, just with the adherent portion 120 having an adhesive applied to one side thereof (the side that will be on the skin during use). The central non-adherent portion 110 and the adherent portion 120 can alternatively be a different material from each other. The dressing material for the central non-adherent portion 110 and/or the adherent portion 120 can be a soft material, such as a soft polymer dressing material. The adherent portion 120 can surround a perimeter of the central non-adherent portion 110. The therapeutic dressing 100 can include at least one stimulating electrode 140 (e.g., a positive stimulating electrode and a negative stimulating electrode), a sensor 160, and an integrated circuit (IC) 150 in operable communication with (e.g., wired or wirelessly), and configured to control, the sensor 160 and the at least one stimulating electrode 140. The IC can include at least one battery to power the IC. The sensor can be, for example, an EMG sensor configured to sense EMG signals (e.g., from a muscle) during use. The therapeutic dressing 100 can include a perforated border 170 between the central non-adherent portion 110 and the adherent portion 120, configured to allow detachment adherent portion 120 (including the IC 150, electrode(s) 140, and sensor 160) while leaving the wound dressing (i.e., the central non-adherent portion 110 undisturbed).

The therapeutic dressing 100 can include an indicator 130 configured to indicate when certain functions are being performed and/or when they are completed or not being performed. For example, the indicator 130 can confirm completion of a successful stimulation action between the nerve and the muscle during use of the therapeutic dressing 100. The indicator 130 can be in operable communication (e.g., wired or wirelessly) with the IC 150, the sensor 160, and/or the electrode(s) 140. While not necessary to the function of the therapeutic dressing 100 (or any part thereof), the indicator 130 can provide supportive information to a treating clinician and/or a user. The indicator 130 can be, for example, one or more lights that illuminate different colors and/or patterns (e.g., blinking, solid) to provide different indications of what is happening with the IC 150, the sensor 160, and/or the electrode(s) 140. The lights can be, for example, light-emitting diodes (LEDs) and/or an LED functional circuit.

The IC 150, the sensor 160, the electrode(s) 140, and the indicator 130 (if present) can all be disposed on the adherent portion 120 of the therapeutic dressing 100. The positions of these elements on the adherent portion 120 can vary, with the positions in FIG. 1 being for exemplary purposes only. However, the IC 150 must be in operable communication with the sensor 160 and the electrode(s) 140; the sensor 160 can be configured to sit over a muscle of interest, and the electrode(s) 140 can be configured to stimulate one or more nerves of interest.

The therapeutic dressing 100 can comprise two layers of material, which can be insulating. That is, the dressing material can be provided in two layers, in the central non-adherent portion 110, in the adherent portion 120, or both. The IC 150 can be provided between the two layers of dressing material, such that it is insulated by the dressing material. The therapeutic dressing 100 can be configured such that the electrode(s) 140 and/or the sensor 160 come into direct physical contact with the skin during use. That is, electrode(s) 140 and/or the sensor 160 can be exposed out of the dressing material on at least one surface (i.e., the bottom surface that comes into contact with the skin during use) and optionally out of both surfaces (i.e., also out of the top surface) of the therapeutic dressing 100.

The dressing material(s) of the therapeutic dressing 100 can be disposable, as can be the remaining components (e.g., the IC 150, the sensor 160, the electrode(s) 140, and the indicator 130 (if present)). Thus, the therapeutic dressing 100 can be specifically designed and configured for a single use (i.e., to be disposable). That is, the therapeutic dressing 100 can be fabricated using low-cost materials such that someone skilled in the art would understand it is intended for a single-use and is not “disposable” only in the sense that anything can theoretically be disposed of.

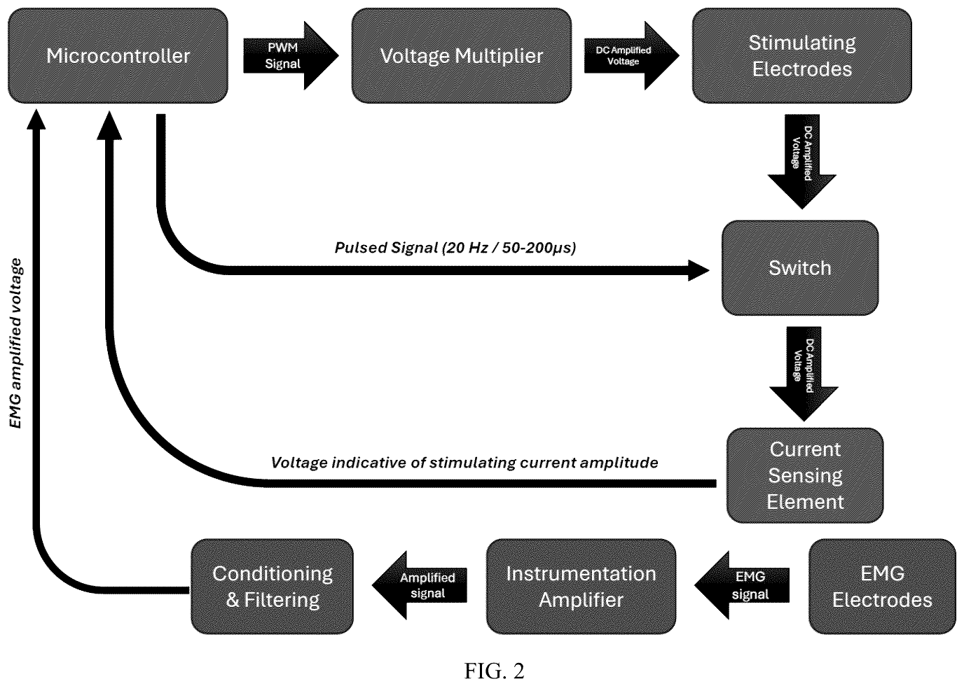

FIG. 2 shows circuit diagram of an IC 150 that can be used with a therapeutic dressing 100, according to an embodiment of the subject invention. Referring to FIG. 2, the circuit can be powered by an onboard power source (e.g., one or more coin/button cell batteries). The IC 150 can comprise a microcontroller configured to generate a pulse-width modulated (PWM) waveform, which can be supplied to a voltage multiplier circuit including capacitive and diode-based stages or a boost circuit. The frequency of the PWM signal can be varied to regulate the cumulative output voltage of the multiplier over a defined time interval. This output voltage can then be used to produce stimulating electrical pulses.

The microcontroller can also generate timed control pulses that drive a switching element, such as a transistor. This switch can be arranged in series with the output of the voltage multiplier, a current-sensing resistor, and a at least one stimulating electrode (e.g., a pair of stimulating electrodes). The voltage developed across the current-sensing resistor, indicative of the stimulation current, can be fed back to the microcontroller. Based on this feedback, the microcontroller can dynamically adjust the PWM frequency to ensure that the stimulation current remains within a desired range. For example, if the sensed current is too low, the frequency may be increased to raise the stimulation voltage, and conversely, reduced when the current is too high.

The system can also include an electromyography (EMG) sensing circuit, which may comprise one or more amplification stages and active filters (e.g., high-pass, low-pass, and/or notch filters). The filtered EMG signal can be rectified and processed to yield a voltage representing muscle activity. This signal can be sampled by the microcontroller, which can then modulate the stimulation parameters (e.g., frequency, duration, and/or amplitude) to achieve a target EMG response.

The system can be controlled by embedded software or firmware. Program code may reside within the microcontroller or be transmitted from an external computing device. This code can implement feedback control, waveform shaping, and/or safety monitoring. The described functionality can be embodied as data and instructions stored in a machine-readable medium, executable by the processor to perform the required operations

The electrode(s) 140 and/or the IC 150 can be configured such that an electrical stimulus (e.g., a constant electrical stimulus) can be delivered to one or more nerves of interest during use (by applying the stimulus to the skin). The IC 150 can include a voltage control mechanism to ensure that the nerve receives the same amount of electrical stimulation regardless of patient/user size or local tissue resistance.

When there has been degeneration of axons within a nerve, electrical stimulation delivered to the nerve (e.g., even for a short time such as one hour) can improve the rate and extent of nerve regeneration. Embodiments of the subject invention provide methods of using a therapeutic dressing 100 as described herein. The therapeutic dressing 100 can be disposed on or over a site of interest on a patient/user (e.g., a surgical site following nerve repair/reconstruction or decompression surgery). The therapeutic dressing 100 can be configured to maintain electrical stimulation for a set number of minutes before becoming inert. The duration of stimulation can be predetermined, for example based on the specific surgical procedure and/or patient/user characteristics. The stimulation can last for an amount of time in a range of, for example, 1 minute to 600 minutes (or any value, about any value, or subrange therein, such as in a range of from 30 minutes to 120 minutes, 30 minutes to 90 minutes, or 45 minutes to 75 minutes). In some embodiments, the stimulation can last for 60 minutes or about 60 minutes. Although a therapeutic dressing for a wound site has been described extensively herein, the device could be affixed to the skin over the anatomical location of a nerve where there is no post operative wound (such that the therapeutic dressing would no longer function as a wound dressing) and perhaps in such a situation could be made more compact, omitting the actual wound coverage part (i.e., the central non-adherent portion 110) of the dressing 100 entirely.

In some embodiments the wound coverage part can be separated from the stimulation part by the use of two layers attached by an adhesive that allows separation by the user if desired or required. That is, the at least one stimulating electrode 140, the sensor 160, and the circuit 150 can be provided on a separate layer (i.e., on an upper layer) from the non-adherent portion 110 and the adherent portion 120 and can be joined thereto by a peelable adhesive, such that a user can peel off the upper layer (with the at least one stimulating electrode 140, the sensor 160, and the circuit 150) while leaving the lower layer 110,120 on the wound as a wound dressing.

In some embodiments, the IC 150 can be controlled by software and/or code. That is, the IC 150 can be connected to a computing device having software installed thereon that can be used to control the IC 150 and/or can be transferred onto the IC 150 (e.g., onto the microcontroller) to control it.

In certain embodiments, the therapeutic dressing 100 can comprise a wireless communication module (e.g., a Bluetooth IC) enabling (and/or configured for) remote adjustment of stimulation parameters and physiological signal monitoring. A digital signal processor (DSP) can be included to condition physiological input signals (e.g. EMG or nerve conduction signals), improve signal fidelity, and reduce artifacts (including junction-reference artifacts).

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

When the term module is used herein, it can refer to software and/or one or more algorithms to perform the function of the module; alternatively, the term module can refer to a physical device configured to perform the function of the module (e.g., by having software and/or one or more algorithms stored thereon).

When ranges are used herein, combinations and subcombinations of ranges (including any value or subrange contained therein) are intended to be explicitly included. When the term “about” or “approximately” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A therapeutic dressing, comprising:
a central non-adherent portion;
an adherent portion around the central non-adherent portion and comprising an adhesive;
at least one stimulating electrode;
a sensor; and
an integrated circuit (IC) in operable communication with, and configured to control, the at least one stimulating electrode,
wherein the therapeutic dressing is configured to provide electrical stimulation to a nerve of a user during use via the at least one stimulating electrode.

2. The therapeutic dressing according to claim 1, wherein the IC is configured such that the nerve receives the same amount of stimulating current regardless of a size of the user or a resistance of skin or tissue of the user, and
wherein the IC comprises a microcontroller, a power source, and a current-controlled voltage pulse generator configured to provide the same amount of stimulating current to the nerve regardless of the size of the user or the resistance of the skin or tissue of the user.

3. The therapeutic dressing according to claim 1, wherein the therapeutic dressing is configured to provide the electrical stimulation to the nerve for a predetermined amount of time before becoming inert.

4. The therapeutic dressing according to claim 3, wherein the predetermined amount of time is based on: characteristics of a wound of the nerve of the user; characteristics of the user; or both, and
wherein the predetermined amount of time is in a range of from 2 minutes to 180 minutes.

5. The therapeutic dressing according to claim 1, further comprising a first layer of a dressing material and a second layer of the dressing material,
wherein the IC is sandwiched between the first layer of the dressing material and the second layer of the dressing material,
wherein the sensor is exposed out of the first layer of the dressing material,
wherein the at least one stimulating electrode is exposed out of the first layer of the dressing material, and
wherein the dressing material is electrically insulating.

6. The therapeutic dressing according to claim 5, wherein the dressing material is a polymer dressing material.

7. The therapeutic dressing according to claim 1, wherein the therapeutic dressing is configured such that the sensor is in direct physical contact with skin of the user during use, and
wherein the therapeutic dressing is configured such that the at least one stimulating electrode is in direct physical contact with skin of the user during use.

8. The therapeutic dressing according to claim 1, wherein the therapeutic dressing is configured such that the sensor is disposed over a muscle of the user during use, and
wherein the sensor is an electromyography (EMG) sensor.

9. The therapeutic dressing according to claim 1, wherein the at least one stimulating electrode comprises a positive stimulating electrode and a negative stimulating electrode.

10. The therapeutic dressing according to claim 1, further comprising an indicator configured to indicate a function of the IC and/or the at least one stimulating electrode during use,
wherein the indicator is disposed in the non-adherent portion, and
wherein the IC, the sensor, and the at least one stimulating electrode are disposed in the non-adherent portion.

11. The therapeutic dressing according to claim 1, wherein the therapeutic dressing is a single-use, disposable dressing.

12. The therapeutic dressing according to claim 1, wherein the IC comprises at least one battery and a voltage multiplier, and
wherein the IC is in operable communication with, and configured to control, the sensor.

13. The therapeutic dressing according to claim 1, wherein the IC comprises a wireless communication module configured to enable remote modification of one or more stimulation parameters of the electrical stimulation.

14. The therapeutic dressing according to claim 13, wherein the wireless communication module comprises a Bluetooth integrated circuit.

15. The therapeutic dressing according to claim 1, wherein the IC further comprises a digital signal processor (DSP) configured to condition one or more physiological input signals, and
wherein the DSP is configured to reduce or eliminate junction-reference artifacts.

16. A method for providing electrical stimulation to a nerve of a user, the method comprising:
applying, to skin of the user, the therapeutic dressing according to claim 1; and
using the therapeutic dressing to provide the electrical stimulation to the nerve for a predetermined amount of time.

17. The method according to claim 16, wherein the predetermined amount of time is based on: characteristics of a wound of the nerve of the user; characteristics of the user; or both, and
wherein the predetermined amount of time is in a range of from 2 minutes to 180 minutes.

18. The method according to claim 16, further comprising disposing of the therapeutic dressing after a single use of the therapeutic dressing providing the electrical stimulation to the nerve for the predetermined amount of time.

19. A method of monitoring a post-operative treatment site, the method comprising:
applying to the post-operative treatment site a therapeutic dressing comprising a circuit and at least one sensor;
receiving a physiological signal from the at least one sensor of the therapeutic dressing;
transmitting the physiological signal wirelessly to a remote device; and
adjusting one or more stimulation parameters of the therapeutic dressing based on the physiological signal.

20. A method for providing electrical stimulation to a nerve of a user, the method comprising:
applying, to skin of the user, a therapeutic dressing comprising at least one stimulating electrode, a sensor, and an integrated circuit (IC) in operable communication with, and configured to control, the at least one stimulating electrode;
receiving and utilizing EMG signal feedback to confirm neural stimulation;
adjusting stimulation parameters of the therapeutic dressing, based on the EMG signal feedback, to deliver or maintain optimal stimulation according to a predetermined protocol.
